(19) United States
(12) Patent Application Publication
(10) Pub. No.: US 2025/0352600 A1
(43) Pub. Date: Nov. 20, 2025

(54) DIETARY SUPPLEMENTS FOR BRAIN HEALTH

(71) Applicant: Melaleuca, Inc., Idaho Falls, ID (US)

(72) Inventors: Jordan Joy, Idaho Falls, ID (US); Nasser A. Fredj, Idaho Falls, ID (US)

(21) Appl. No.: 19/205,421

(22) Filed: May 12, 2025

Related U.S. Application Data
(60) Provisional application No. 63/647,878, filed on May 15, 2024.

(51) Int. Cl. A61K 36/74; A61K 31/198; A61K 31/522; A61P 25/00; A61P 25/26; A61P 25/28

(52) U.S. Cl. CPC A61K 36/742; A61K 31/198; A61K 31/522; A61P 25/00; A61P 25/26; A61P 25/28

(57) ABSTRACT

Brain health compositions are described herein. For example, this document provides brain health compositions containing a Coffea extract and/or dried Coffea (e.g., a coffeeberry extract), caffeine (e.g., instant release caffeine and/or sustained release caffeine), theacrine, and methylliberine. Such compositions may be used to, e.g., help improve executive functioning, reduce testing errors, and/or provide even greater improvement in general alertness, attention, brain power, and reaction time over caffeine alone.

### CROSS-REFERENCE TO RELATED APPLICATIONS

This application claims priority from U.S. Provisional Application No. 63/647,878, filed on May 15, 2024. The disclosure of the prior application is considered part of (and is incorporated by reference in) the disclosure of this application.

### TECHNICAL FIELD

This document relates to the field of brain health compositions. For example, this document relates to brain health compositions useful for, for example, one or more of reducing fatigue, improving attention, improving alertness, improving working memory, and supporting mood.

### BACKGROUND

The world we live in today feels busier and more rushed than ever. Working professionals must reach numerous deadlines and hit elevated performance milestones set by employers. Higher education has increased the pressure on students to reach higher and higher standards in the classroom and the general stresses and demands of life are getting more and more strenuous. These modern stresses have created an entire market of medicines, supplements, and drinks to aid in focus. The current market for focus support supplements includes ADHD medications like ADDERALL® or CONCERTA® and over the counter brain stimulants like highly caffeinated drinks, mushroom supplements, or herbal remedies. However, classic ADHD medications like ADDERALL® can have severe side effects including insomnia, nausea, anxiety, and irritability, which can be further detrimental to someone dealing with focus issues. Over the counter options with high doses of caffeine can assist in focus, even for people with ADHD, but high doses of caffeine can also have problematic side effects. For example, high doses of caffeine can cause jitters, nervousness, restlessness, anxiety, insomnia, and increased heart rate as well as be dependence forming. The efficacy of mushroom supplements is spotty at best. There is a continuing need to formulate compositions that support and/or improve, for example, focus, mood, and alertness.

### SUMMARY

Provided herein are brain health compositions comprising a Coffea extract and/or dried Coffea and one or more of caffeine, theacrine, methylliberine, and theanine. Also provided herein are brain health compositions comprising a coffeeberry extract and one or more of caffeine, theacrine, methylliberine, and theanine.

In some embodiments, the brain health composition comprises the coffeeberry extract, caffeine, and two or more of theacrine, methylliberine, and theanine.

Also provided herein are brain health compositions comprising: caffeine; a coffeeberry extract; theacrine; and methylliberine.

In some embodiments, the caffeine is present in an amount of about 60 mg to about 200 mg per serving of the brain health composition. In some embodiments, the caffeine is present in an amount of about 140 mg to about 200 mg per serving of the brain health composition. In some embodiments, wherein the caffeine is present in an amount of about 180 mg per serving of the brain health composition.

In some embodiments, the caffeine comprises instant release caffeine and sustained release caffeine. In some embodiments, the instant release caffeine is present in an amount of about 60 mg to about 200 mg per serving of the brain health composition. In some embodiments, the instant release caffeine is present in an amount of about 60 to about 140 mg per serving of the brain health composition. In some embodiments, the instant release caffeine is anhydrous caffeine.

In some embodiments, the sustained release caffeine is present in an amount of about 50 mg to about 200 mg per serving of the brain health composition. In some embodiments, the sustained release caffeine is present in an amount of about 40 mg to about 80 per serving of the brain health composition. In some embodiments, the instant release caffeine and the sustained release caffeine are present in an amount of about 180 mg per serving of the brain health composition.

In some embodiments, the sustained release caffeine is released over a period of about 2 to about 12 hours when consumed by a mammal. In some embodiments, the sustained release caffeine exhibits a release lag time of about 2 to about 4 hours followed by complete release by about 6 to about 8 hours when consumed by a mammal.

In some embodiments, the coffeeberry extract is present in an amount of about 50 mg to about 250 mg per serving of the brain health composition. In some embodiments, the coffeeberry extract is present in an amount of about 75 mg to about 125 mg per serving of the brain health composition. In some embodiments, the coffeeberry extract is present in an amount of about 100 mg per serving of the brain health composition.

In some embodiments, the coffeeberry extract is a Coffea arabica fruit extract.

In some embodiments, the theacrine is present in an amount of about 40 mg to about 100 mg per serving of the brain health composition. In some embodiments, the theacrine is present in an amount of about 40 mg to about 80 mg per serving of the brain health composition.

In some embodiments, the methylliberine is present in an amount of about 40 mg to about 100 mg per serving of the brain health composition. In some embodiments, the methylliberine is present in an amount of about 50 mg to about 90 mg per serving of the brain health composition.

In some embodiments, the theacrine and the methylliberine are present in an amount of about 125 mg per serving of the brain health composition.

In some embodiments, the brain health composition is formulated as a tablet, a capsule, or a soft gel. In some embodiments, one tablet, capsule, or soft gel comprises a full serving of the brain health composition.

In some embodiments, the brain health composition is formulated as a capsule.

Also provided herein are methods of improving one or more of alertness, reaction time, attention, mental accuracy, working memory, and video game performance in a mammal, comprising administering to the mammal any of the brain health composition provided herein.

In some embodiments, one or more of alertness, reaction time, attention, mental accuracy, working memory, and video game performance are improved after administration of the brain health composition as compared to before administration of the composition.

Also provided herein are methods of reducing one or more of fatigue, anxiety, occasional feelings of stress, and test errors in a mammal, comprising administering to the mammal any of the brain health composition provided herein.

In some embodiments, one or more of fatigue, anxiety, occasional feelings of stress, and test errors are reduced after administration of the brain health composition as compared to before administration of the composition.

Also provided herein methods of supporting one or more of healthy energy levels, mood, and brain function in a mammal, comprising administering to the mammal any of the brain health composition provided herein.

In some embodiments, one or more of healthy energy levels, mood, and brain function are supported after administration of the brain health composition as compared to before administration of the composition.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a plot showing fatigue pre and post consumption of Composition A; * indicates p<0.05 from pre.

FIG. 2 is a plot showing vigor pre and post consumption of Composition A; * indicates p<0.05 from pre.

FIG. 3 is a plot showing TMD pre and post consumption of Composition A; * indicates p<0.05 from pre.

FIG. 4 is a plot showing processing speed pre and post consumption of Composition A; * indicates p<0.05 from pre.

FIG. 5 is a plot showing executive function pre and post consumption of Composition A; * indicates p<0.05 from pre.

### DETAILED DESCRIPTION

This document provides brain health compositions that contain a Coffea extract and/or dried Coffea (e.g., a coffeeberry extract) and one or more of caffeine (e.g., instant release caffeine and/or sustained release caffeine), theacrine, methylliberine, and theanine. For example, in some embodiments, a brain health composition provided herein contains a Coffea extract and/or dried Coffea (e.g., a coffeeberry extract), caffeine (e.g., instant release caffeine and/or sustained release caffeine), theacrine, and methylliberine. In some embodiments, a brain health composition provided herein contains a Coffea extract and/or dried Coffea (e.g., a coffeeberry extract), caffeine (e.g., instant release caffeine and/or sustained release caffeine), and theanine. In some embodiments, a brain health composition provided herein contains a Coffea extract and/or dried Coffea (e.g., a coffeeberry extract), caffeine (e.g., instant release caffeine and/or sustained release caffeine), methylliberine, and theanine. In some embodiments, a brain health composition provided herein contains a Coffea extract and/or dried Coffea (e.g., a coffeeberry extract), caffeine (e.g., instant release caffeine and/or sustained release caffeine), theacrine, methylliberine, and theanine.

The brain health compositions provided herein can advantageously release caffeine gradually to provide energy for an extended period without causing caffeine jitters or crashes. The combination of, for example, caffeine with theacrine and/or methylliberine can enhance the effects of caffeine while mitigating the unwanted side effects. Coffeeberry (e.g., a coffeeberry extract) can help form new connections between neurons supporting better learning and memory. Accordingly, the brain health compositions provided herein can be used, for example, to provide sustained energy to support one or more of mental energy, working memory, and performance at work or school. In some embodiments, the brain health compositions provided herein can help improve executive functioning, reduce testing errors, and/or provide even greater improvement in general alertness, brain power, and reaction time over caffeine alone.

### Compositions

### Coffea

The brain health compositions provided herein include a Coffea extract and/or dried Coffea. In various embodiments, the brain health compositions provided herein can include a Coffea extract and/or dried Coffea and one or more of caffeine, theacrine, methylliberine, and theanine.

A Coffea extract and/or dried Coffea can be obtained from one or more members of the Coffea genus. Non-limiting examples of members of the Coffea genus include Coffea arabica and Coffea canephora. Any appropriate method can be used to obtain a Coffea extract and/or dried Coffea for use in the brain health compositions provided herein. For example, an aqueous and/or ethanolic solution can be used to obtain an Coffea extract from one or more of: Coffea roots, Coffea stems, Coffea leaves, Coffea flowers, Coffea fruit, Coffea seeds, and Coffea petioles. A Coffea extract can be in the form of a liquid (e.g., an aqueous solution). In some embodiments, a Coffea extract is a Coffea arabica extract. For example, a Coffea arabica can be an extract from one or more of: Coffea arabica roots, Coffea arabica stems, Coffea arabica leaves, Coffea arabica flowers, Coffea arabica fruit, Coffea arabica seeds, and Coffea arabica petioles.

Dried Coffea can include one or more of: dried Coffea roots, dried Coffea stems, dried Coffea leaves, dried Coffea flowers, dried Coffea fruit, dried Coffea seeds, and dried Coffea petioles. Dried Coffea can be in the form of a powder.

The brain health compositions provided herein can contain any appropriate amount of a Coffea extract and/or dried Coffea. In some embodiments, a Coffea extract and/or dried Coffea) is present in an amount of at least about 1 mg per serving of a brain health composition provided herein (e.g., at least about 10 mg, at least about 25 mg, at least about 50 mg, at least about 75 mg, at least about 100 mg, at least about 125 mg, at least about 150 mg, at least about 200 mg, at least about 250 mg, or at least about 300 mg per serving of a brain health composition provided herein). In some embodiments, a Coffea extract and/or dried Coffea is present in an amount of no more than about 525 mg per serving of a brain health composition provided herein (e.g., no more than about 500 mg, no more than about 400 mg, no more than about 375 mg, no more than about 350 mg, no more than about 325 mg, no more than about 300 mg, no more than about 250 mg, no more than about 200 mg, no more than about 150 mg, or no more than about 100 mg per serving of a brain health composition provided herein).

In some embodiments, a Coffea extract and/or dried Coffea is present in an amount of about 10 mg to about 500 mg per serving of a brain health composition provided herein. For example, a Coffea extract and/or dried Coffea can be present in an amount of about 10 mg to about 50 mg, about 10 mg to about 100 mg, about 10 mg to about 150 mg, about 10 mg to about 200 mg, about 10 mg to about 250 mg, about 10 mg to about 300 mg, about 10 mg to about 350 mg, about 10 mg to about 400 mg, about 10 mg to about 450 mg, about 50 mg to about 100 mg, about 50 mg to about 150 mg, about 50 mg to about 200 mg, about 50 mg to about 250 mg, about 50 mg to about 300 mg, about 50 mg to about 350 mg, about 50 mg to about 400 mg, about 50 mg to about 450 mg, about 50 mg to about 500 mg, about 100 mg to about 150 mg, about 100 mg to about 200 mg, about 100 mg to about 250 mg, about 100 mg to about 300 mg, about 100 mg to about 350 mg, 100 mg to about 400 mg, about 100 mg to about 450 mg, about 100 mg to about 500 mg, about 150 mg to about 200 mg, about 150 mg to about 250 mg, about 150 mg to about 300 mg, about 150 mg to about 350 mg, 150 mg to about 400 mg, about 150 mg to about 450 mg, about 150 mg to about 500 mg, about 200 mg to about 250 mg, about 200 mg to about 300 mg, about 200 mg to about 350 mg, 200 mg to about 400 mg, about 200 mg to about 450 mg, about 200 mg to about 500 mg, about 250 mg to about 300 mg, about 250 mg to about 350 mg, 250 mg to about 400 mg, about 250 mg to about 450 mg, about 250 mg to about 500 mg, about 300 mg to about 350 mg, 300 mg to about 400 mg, about 300 mg to about 450 mg, about 300 mg to about 500 mg, 350 mg to about 400 mg, about 350 mg to about 450 mg, about 350 mg to about 500 mg, about 400 mg to about 450 mg, about 400 mg to about 500 mg, or about 450 mg to about 500 mg per serving of a brain health composition provided herein. In some embodiments, a Coffea extract and/or dried Coffea is present in an amount of about 50 to about 250 mg per serving of a brain health composition provided herein. In some embodiments, a Coffea extract and/or dried Coffea is present in an amount of about 75 to about 125 mg per serving of a brain health composition provided herein.

In some embodiments, a Coffea extract and/or dried Coffea is present in an amount of about 10 mg, about 25 mg, about 50 mg, about 75 mg, about 90 mg, about 100 mg, about 110 mg, about 125 mg, about 175 mg, about 250 mg, about 300 mg, about 400 mg, or about 500 mg per serving of a brain health composition provided herein. In some embodiments, a Coffea extract and/or dried Coffea is present in an amount of about 100 mg per serving of a brain health composition provided herein.

In some embodiments, a Coffea extract and/or dried Coffea is present in an amount of about 2% to about 60% w/w of a brain health composition provided herein. For example, coffeeberry can be present in an amount of about 2% to about 10%, about 2% to about 20%, about 2% to about 30%, about 2% to about 40%, about 2% to about 50%, about 10% to about 20%, about 10% to about 30%, about 10% to about 40%, about 10% to about 50%, about 10% to about 60%, about 20% to about 30%, about 20% to about 40%, about 20% to about 50%, about 20% to about 60%, about 30% to about 40%, about 30% to about 50%, about 30% to about 60%, about 40% to about 50%, about 40% to about 60%, or about 50% to about 60% w/w of a brain health composition provided herein. In some embodiments, a Coffea extract and/or dried Coffea is present in an amount of about 10% to about 30%/w of a brain health composition provided herein.

In some embodiments, a Coffea extract and/or dried Coffea is present in an amount of about 2%, about 10%, about 12%, about 14%, about 16% about 18%, about 20%, about 22%, about 24%, about 26%, about 28%, about 30%, about 40%, about 50%, or about 60% w/w of a brain health composition provided herein. In some embodiments, a Coffea extract and/or dried Coffea is present in an amount of about 20% w/w of a brain health composition provided herein.

In some embodiments, a Coffea extract and/or a Coffea powder in a brain health composition provided herein contains coffeeberry. Coffeeberry can refer to the fruit of a Coffea plant (also referred to as a coffee cherry). Coffeeberry can increase brain-derived neurotrophic factor (BDNF), a protein in the brain that can help form new connections between neurons and support better learning and memory. In some embodiments, coffeeberry increases neuroplasticity. In various embodiments, the brain health compositions provided herein can include coffeeberry and one or more of caffeine, theacrine, methylliberine, and theanine.

In some embodiments, a brain health composition provided herein can include dried coffeeberry (e.g., as whole dried coffeeberries, powdered dried coffeeberries, and/or granulized dried coffeeberries) and/or a coffeeberry extract. A coffeeberry extract can be in the form of a liquid (e.g., an aqueous solution), a solid (e.g., a particulate or a powder), or a semi-solid form (e.g., a gel or an emulsion). Any appropriate method can be used to obtain coffeeberry for use in the brain health compositions provided herein. For example, a coffeeberry extract can be obtained by aqueous and/or ethanolic extraction from fruits from one or more members of the Coffea genus. In some embodiments, a coffeeberry extract is an extract prepared from the fruits of C. arabica.

The brain health compositions provided herein can contain any appropriate amount of coffeeberry. In some embodiments, coffeeberry (e.g., a coffeeberry extract) is present in an amount of at least about 1 mg per serving of a brain health composition provided herein (e.g., at least about 10 mg, at least about 25 mg, at least about 50 mg, at least about 75 mg, at least about 100 mg, at least about 125 mg, at least about 150 mg, at least about 200 mg, at least about 250 mg, or at least about 300 mg per serving of a brain health composition provided herein). In some embodiments, coffeeberry (e.g., a coffeeberry extract) is present in an amount of no more than about 525 mg per serving of a brain health composition provided herein (e.g., no more than about 500 mg, no more than about 400 mg, no more than about 375 mg, no more than about 350 mg, no more than about 325 mg, no more than about 300 mg, no more than about 250 mg, no more than about 200 mg, no more than about 150 mg, or no more than about 100 mg per serving of a brain health composition provided herein).

In some embodiments, coffeeberry (e.g., a coffeeberry extract) is present in an amount of about 10 mg to about 500 mg per serving of a brain health composition provided herein. For example, coffeeberry can be present in an amount of about 10 mg to about 50 mg, about 10 mg to about 100 mg, about 10 mg to about 150 mg, about 10 mg to about 200 mg, about 10 mg to about 250 mg, about 10 mg to about 300 mg, about 10 mg to about 350 mg, about 10 mg to about 400 mg, about 10 mg to about 450 mg, about 50 mg to about 100 mg, about 50 mg to about 150 mg, about 50 mg to about 200 mg, about 50 mg to about 250 mg, about 50 mg to about 300 mg, about 50 mg to about 350 mg, about 50 mg to about 400 mg, about 50 mg to about 450 mg, about 50 mg to about 500 mg, about 100 mg to about 150 mg, about 100 mg to about 200 mg, about 100 mg to about 250 mg, about 100 mg to about 300 mg, about 100 mg to about 350 mg, 100 mg to about 400 mg, about 100 mg to about 450 mg, about 100 mg to about 500 mg, about 150 mg to about 200 mg, about 150 mg to about 250 mg, about 150 mg to about 300 mg, about 150 mg to about 350 mg, 150 mg to about 400 mg, about 150 mg to about 450 mg, about 150 mg to about 500 mg, about 200 mg to about 250 mg, about 200 mg to about 300 mg, about 200 mg to about 350 mg, 200 mg to about 400 mg, about 200 mg to about 450 mg, about 200 mg to about 500 mg, about 250 mg to about 300 mg, about 250 mg to about 350 mg, 250 mg to about 400 mg, about 250 mg to about 450 mg, about 250 mg to about 500 mg, about 300 mg to about 350 mg, 300 mg to about 400 mg, about 300 mg to about 450 mg, about 300 mg to about 500 mg, 350 mg to about 400 mg, about 350 mg to about 450 mg, about 350 mg to about 500 mg, about 400 mg to about 450 mg, about 400 mg to about 500 mg, or about 450 mg to about 500 mg per serving of a brain health composition provided herein. In some embodiments, coffeeberry is present in an amount of about 50 to about 250 mg per serving of a brain health composition provided herein. In some embodiments, coffeeberry is present in an amount of about 75 to about 125 mg per serving of a brain health composition provided herein.

In some embodiments, coffeeberry (e.g., a coffeeberry extract) is present in an amount of about 10 mg, about 25 mg, about 50 mg, about 75 mg, about 90 mg, about 100 mg, about 110 mg, about 125 mg, about 175 mg, about 250 mg, about 300 mg, about 400 mg, or about 500 mg per serving of a brain health composition provided herein. In some embodiments, coffeeberry is present in an amount of about 100 mg per serving of a brain health composition provided herein.

In some embodiments, coffeeberry (e.g., a coffeeberry extract) is present in an amount of about 2% to about 60% w/w of a brain health composition provided herein. For example, coffeeberry can be present in an amount of about 2% to about 10%, about 2% to about 20%, about 2% to about 30%, about 2% to about 40%, about 2% to about 50%, about 10% to about 20%, about 10% to about 30%, about 10% to about 40%, about 10% to about 50%, about 10% to about 60%, about 20% to about 30%, about 20% to about 40%, about 20% to about 50%, about 20% to about 60%, about 30% to about 40%, about 30% to about 50%, about 30% to about 60%, about 40% to about 50%, about 40% to about 60%, or about 50% to about 60% w/w of a brain health composition provided herein. In some embodiments, coffeeberry is present in an amount of about 10% to about 30%/w of a brain health composition provided herein. In some embodiments, coffeeberry is present in an amount of about 2%, about 10%, about 12%, about 14%, about 16% about 18%, about 20%, about 22%, about 24%, about 26%, about 28%, about 30%, about 40%, about 50%, or about 60% w/w of a brain health composition provided herein. In some embodiments, coffeeberry is present in an amount of about 20% w/w of a brain health composition provided herein.

### Caffeine

The brain health compositions provided herein include caffeine. Caffeine is a natural stimulant that can increase alertness, enhance cognitive performance, and/or improve focus and attention. For example, caffeine may increase alertness and decrease fatigue by inhibiting the action of adenosine at the adenosine receptor as well as acting as a beta-adrenergic receptor to release adrenaline.

Any appropriate method can be used to obtain caffeine for use in the brain health compositions provided herein. For example, caffeine can be obtained by extraction (e.g., water, supercritical carbon dioxide, or organic solvent extraction) of caffeine from coffee and/or by chemical synthesis. In some embodiments, the caffeine is from a green coffee extract. In some embodiments, the caffeine is from an extract of Coffea arabica beans.

In some embodiments, the caffeine is anhydrous caffeine. In some embodiments, the caffeine is not formulated for sustained release. For example, the caffeine can be instant release caffeine.

The brain health compositions provided herein can contain any appropriate amount of caffeine (e.g., anhydrous and/or instant release caffeine). In some embodiments, caffeine (e.g., anhydrous and/or instant release caffeine) is present in an amount of at least about 10 mg per serving of a brain health composition provided herein (e.g., at least about 20 mg, at least about 50 mg, at least about 75 mg, at least about 100 mg, at least about 125 mg, at least about 150 mg, at least about 200 mg, or at least about 250 mg per serving of a brain health composition provided herein). In some embodiments, caffeine (e.g., anhydrous and/or instant release caffeine) is present in an amount of no more than about 425 mg per serving of a brain health composition provided herein (e.g., no more than about 400 mg, no more than about 375 mg, no more than about 350 mg, no more than about 325 mg, no more than about 300 mg, no more than about 250 mg, or no more than about 200 mg per serving of a brain health composition provided herein).

In some embodiments, caffeine (e.g., anhydrous and/or instant release caffeine) is present in an amount of about 20 mg to about 400 mg per serving of a brain health composition provided herein. For example, caffeine can be present in an amount of about 20 mg to about 50 mg, about 20 mg to about 75 mg, about 20 mg to about 100 mg, about 20 mg to about 125 mg, about 20 mg to about 150 mg, about 20 mg to about 175 mg, about 20 mg to about 200 mg, about 20 mg to about 225 mg, about 20 mg to about 250 mg, about 20 mg to about 275 mg, about 20 mg to about 300 mg, about 20 mg to about 325 mg, about 20 mg to about 350 mg, about 20 mg to about 375 mg, about 50 mg to about 75 mg, about 50 mg to about 100 mg, about 50 mg to about 125 mg, about 50 mg to about 150 mg, about 50 mg to about 175 mg, about 50 mg to about 200 mg, about 50 mg to about 225 mg, about 50 mg to about 250 mg, about 50 mg to about 275 mg, about 50 mg to about 300 mg, about 50 mg to about 325 mg, about 50 mg to about 350 mg, about 50 mg to about 375 mg, about 50 mg to about 400 mg, about 100 mg to about 125 mg, about 100 mg to about 150 mg, about 100 mg to about 175 mg, about 100 mg to about 200 mg, about 100 mg to about 225 mg, about 100 mg to about 250 mg, about 100 mg to about 275 mg, about 100 mg to about 300 mg, about 100 mg to about 325 mg, about 100 mg to about 350 mg, about 100 mg to about 375 mg, about 100 mg to about 400 mg, about 150 mg to about 175 mg, about 150 mg to about 200 mg, about 150 mg to about 225 mg, about 150 mg to about 250 mg, about 150 mg to about 275 mg, about 150 mg to about 300 mg, about 150 mg to about 325 mg, about 150 mg to about 350 mg, about 150 mg to about 375 mg, about 150 mg to about 400 mg, about 200 mg to about 225 mg, about 200 mg to about 250 mg, about 200 mg to about 275 mg, about 200 mg to about 300 mg, about 200 mg to about 325 mg, about 200 mg to about 350 mg, about 200 mg to about 375 mg, about 200 mg to about 400 mg, about 250 mg to about 275 mg, about 250 mg to about 300 mg, about 250 mg to about 325 mg, about 250 mg to about 350 mg, about 250 mg to about 375 mg, about 250 mg to about 400 mg, about 300 mg to about 325 mg, about 300 mg to about 350 mg, about 300 mg to about 375 mg, about 300 mg to about 400 mg, about 350 mg to about 375 mg, or about 350 mg to about 400 mg per serving of a brain health composition provided herein. In some embodiments, caffeine is present in an amount of about 60 to about 200 mg per serving of a brain health composition provided herein. In some embodiments, caffeine is present in an amount of about 100 to about 140 mg per serving of a brain health composition provided herein. In some embodiments, caffeine is present in an amount of about 60 to about 100 mg per serving of a brain health composition provided herein.

In some embodiments, caffeine (e.g., anhydrous and/or instant release caffeine) is present in an amount of about 50 mg, about 75 mg, about 90 mg, about 100 mg, about 110 mg, about 120 mg, about 130 mg, about 140 mg, about 150 mg, about 175 mg, about 200 mg, about 225 mg, about 250 mg, about 300 mg, or about 400 mg per serving of a brain health composition provided herein.

In some embodiments, caffeine (e.g., anhydrous and/or instant release caffeine) is present in an amount of about 5% to about 75% w/w of a brain health composition provided herein. For example, caffeine can be present in an amount of about 5% to about 10%, about 5% to about 20%, about 5% to about 30%, about 5% to about 40%, about 5% to about 50%, about 5% to about 60%, about 5% to about 70%, about 10% to about 20%, about 10% to about 30%, about 10% to about 40%, about 10% to about 50%, about 10% to about 60%, about 10% to about 70%, about 10% to about 75%, about 20% to about 30%, about 20% to about 40%, about 20% to about 50%, about 20% to about 60%, about 20% to about 70%, about 20% to about 75%, about 30% to about 40%, about 30% to about 50%, about 30% to about 60%, about 30% to about 70%, about 30% to about 75%, about 40% to about 50%, about 40% to about 60%, about 40% to about 70%, about 40% to about 75%, about 50% to about 60%, about 50% to about 70%, about 50% to about 75%, about 60% to about 70%, about 60% to about 75%, or about 70% to about 75% w/w of a brain health composition provided herein. In some embodiments, caffeine is present in an amount of about 15% to about 40% w/w of a brain health composition provided herein. In some embodiments, caffeine is present in an amount of about 5%, about 10%, about 15%, about 20%, about 22%, about 24%, about 26%, about 28%, about 30%, about 35%, about 40%, about 50%, about 60%, about 70%, or about 75% w/w of a brain health composition provided herein. In some embodiments, caffeine is present in an amount of about 24.5% w/w of a brain health composition provided herein.

Caffeine typically has a half-life of about 3 to about 7 hours depending on the rate of caffeine metabolism, which can be influenced by a person's genetics. Users of caffeine often report a “crash” occurring near that time frame after ingestion of a single administration. A formulation for sustained release of caffeine can have tangible improvements in mood that last all day (e.g., about 8 or more hours) compared to instant release caffeine alone. A formulation for sustained release of caffeine can include, for example, shellac, one or more release retarding agents (e.g., one or more hydrophilic substances, one or more lipophilic substances, one or more pH sensitive polymers, one or more fatty substances, one or more waxes, or mixtures thereof), one or more excipients (e.g., any of the excipients described herein), or mixtures thereof. Including both instant release caffeine and a formulation for sustained release of caffeine in a brain health composition provided herein may provide energy quickly while preventing a crash later (e.g., as compared to instant release caffeine alone). In some embodiments, a brain health composition provided herein further includes a formulation for sustained release of caffeine.

In some embodiments, sustained release caffeine (e.g., the caffeine in a formulation for sustained release of caffeine) is present in an amount of at least about 0.1 mg per serving of a brain health composition provided herein (e.g., at least about 1 mg, at least about 50 mg, at least about 75 mg, at least about 100 mg, at least about 125 mg, at least about 150 mg, at least about 200 mg, or at least about 250 mg per serving of a brain health composition provided herein). In some embodiments, sustained release caffeine (e.g., the caffeine in a formulation for sustained release of caffeine) is present in an amount of no more than about 425 mg per serving of a brain health composition provided herein (e.g., no more than about 400 mg, no more than about 375 mg, no more than about 350 mg, no more than about 325 mg, no more than about 300 mg, no more than about 250 mg, or no more than about 200 mg per serving of a brain health composition provided herein).

In some embodiments, sustained release caffeine (e.g., the caffeine in a formulation for sustained release of caffeine) is present in an amount of about 1 mg to about 400 mg per serving of a brain health composition provided herein. For example, sustained release caffeine can be present in an amount of about 1 mg to about 50 mg, about 1 mg to about 75 mg, about 1 mg to about 100 mg, about 1 mg to about 125 mg, about 1 mg to about 150 mg, about 1 mg to about 175 mg, about 1 mg to about 200 mg, about 1 mg to about 225 mg, about 1 mg to about 250 mg, about 1 mg to about 275 mg, about 1 mg to about 300 mg, about 1 mg to about 325 mg, about 1 mg to about 350 mg, about 1 mg to about 375 mg, about 50 mg to about 75 mg, about 50 mg to about 100 mg, about 50 mg to about 125 mg, about 50 mg to about 150 mg, about 50 mg to about 175 mg, about 50 mg to about 200 mg, about 50 mg to about 225 mg, about 50 mg to about 250 mg, about 50 mg to about 275 mg, about 50 mg to about 300 mg, about 50 mg to about 325 mg, about 50 mg to about 350 mg, about 50 mg to about 375 mg, about 50 mg to about 400 mg, about 100 mg to about 125 mg, about 100 mg to about 150 mg, about 100 mg to about 175 mg, about 100 mg to about 200 mg, about 100 mg to about 225 mg, about 100 mg to about 250 mg, about 100 mg to about 275 mg, about 100 mg to about 300 mg, about 100 mg to about 325 mg, about 100 mg to about 350 mg, about 100 mg to about 375 mg, about 100 mg to about 400 mg, about 150 mg to about 175 mg, about 150 mg to about 200 mg, about 150 mg to about 225 mg, about 150 mg to about 250 mg, about 150 mg to about 275 mg, about 150 mg to about 300 mg, about 150 mg to about 325 mg, about 150 mg to about 350 mg, about 150 mg to about 375 mg, about 150 mg to about 400 mg, about 200 mg to about 225 mg, about 200 mg to about 250 mg, about 200 mg to about 275 mg, about 200 mg to about 300 mg, about 200 mg to about 325 mg, about 200 mg to about 350 mg, about 200 mg to about 375 mg, about 200 mg to about 400 mg, about 250 mg to about 275 mg, about 250 mg to about 300 mg, about 250 mg to about 325 mg, about 250 mg to about 350 mg, about 250 mg to about 375 mg, about 250 mg to about 400 mg, about 300 mg to about 325 mg, about 300 mg to about 350 mg, about 300 mg to about 375 mg, about 300 mg to about 400 mg, about 350 mg to about 375 mg, or about 350 mg to about 400 mg per serving of a brain health composition provided herein. In some embodiments, sustained release caffeine is present in an amount of about 50 to about 200 mg per serving of a brain health composition provided herein. In some embodiments, sustained release caffeine is present in an amount of about 60 to about 200 mg per serving of a brain health composition provided herein. In some embodiments, sustained release caffeine is present in an amount of about 40 to about 80 mg per serving of a brain health composition provided herein. In some embodiments, sustained release caffeine is present in an amount of about 60 to about 100 mg per serving of a brain health composition provided herein.

In some embodiments, sustained release caffeine is present in an amount of about 1 mg, about 40 mg, about 50 mg, about 60 mg, about 70 mg, about 80 mg, about 90 mg, about 100 mg, about 110 mg, about 125 mg, about 150 mg, about 200 mg, about 225 mg, about 250 mg, about 300 mg, or about 400 mg per serving of a brain health composition provided herein.

In some embodiments, sustained release caffeine is present in an amount of about 1% to about 75% w/w of a brain health composition provided herein. For example, sustained release caffeine can be present in an amount of about 1 to about 10%, about 1% to about 20%, about 1% to about 30%, about 1% to about 40%, about 1% to about 50%, about 1% to about 60%, about 1% to about 70%, about 10% to about 20%, about 10% to about 30%, about 10% to about 40%, about 10% to about 50%, about 10% to about 60%, about 10% to about 70%, about 10% to about 75%, about 20% to about 30%, about 20% to about 40%, about 20% to about 50%, about 20% to about 60%, about 20% to about 70%, about 20% to about 75%, about 30% to about 40%, about 30% to about 50%, about 30% to about 60%, about 30% to about 70%, about 30% to about 75%, about 40% to about 50%, about 40% to about 60%, about 40% to about 70%, about 40% to about 75%, about 50% to about 60%, about 50% to about 70%, about 50% to about 75%, about 60% to about 70%, about 60% to about 75%, or about 70% to about 75% w/w of a brain health composition provided herein. In some embodiments, sustained release caffeine is present in an amount of about 5% to about 15% w/w of a brain health composition provided herein. In some embodiments, sustained release caffeine is present in an amount of about 1%, about 5%, about 6%, about 7%, about 8%, about 9%, about 10%, about 11%, about 12%, about 13%, about 14%, about 15%, about 20%, about 30%, about 40%, about 50%, about 60%, about 70%, or about 75% w/w of a brain health composition provided herein. In some embodiments, sustained release caffeine is present in an amount of about 12% w/w of a brain health composition provided herein.

In some embodiments, including both instant release caffeine and sustained release caffeine in a brain health composition provided herein supports mental energy and mood for at least about 8 hours. For example, including both instant release caffeine and sustained release caffeine in a brain health composition provided herein can support mental energy and mood for about 8 hours to about 14 hours, about 8 hours to about 12 hours, about 8 hours to about 10 hours, about 10 to about 14 hours, or about 10 to about 12 hours.

In some embodiments, a formulation for sustained release of caffeine is formulated to release the caffeine over a period of about 6 to about 8 hours. In some embodiments, a formulation for sustained release of caffeine is formulated to release the caffeine in multiple pulses over a period of about 2 to about 12 hours. In some embodiments, a formulation for sustained release of caffeine is formulated to release the caffeine in multiple pulses over the gastrointestinal tract. In some embodiments, a formulation for sustained release of caffeine is formulated to exhibit a lag time for release of the caffeine for about 2 to about 4 hours after consuming a brain health composition provided herein followed by complete release of the caffeine about 6 to 8 hours after consuming a brain health composition provided herein. In some embodiments, a formulation for sustained release of caffeine is formulated to release (i) about 30% or less of the caffeine within about 2 hours at a pH of about 1.2; and/or (ii) about 60% or less of the caffeine within about 4 hours at a pH of about 5.5; and/or (iii) about 100% of the caffeine at a pH of about 7.2 in about 8 hours.

In some embodiments, instant release caffeine is present in an amount of about 60 to about 200 mg per serving of a brain health composition provided herein and sustained release caffeine is present in an amount of about 60 to about 200 mg per serving of a brain health composition provided herein. In some embodiments, instant release caffeine is present in an amount of about 100 to about 140 mg per serving of a brain health composition provided herein and sustained release caffeine is present in an amount of about 40 to about 80 mg per serving of a brain health composition provided herein.

In some embodiments, instant release caffeine and sustained release caffeine are present in a brain health composition provided herein at a ratio of about 4:1 to about 1:4 instant release caffeine to sustained release caffeine. For example, instant release caffeine and sustained release caffeine can be present in a brain health composition provided herein at a ratio of about 4:1 to about 3:1, about 4:1 to about 2:1, about 4:1 to about 1:1, about 4:1 to about 1:2, about 4:1 to about 1:3, about 3:1 to about 2:1, about 3:1 to about 1:1, about 3:1 to about 1:2, about 3:1 to about 1:3, about 3:1 to about 1:4, about 2:1 to about 1:1, about 2:1 to about 1:2, about 2:1 to about 1:3, about 2:1 to about 1:4, about 1:1 to about 1:2, about 1:1 to about 1:3, about 1:1 to about 1:4, about 1:2 to about 1:3, about 1:2 to about 1:4, or about 1:3 to about 1:4 instant release caffeine to sustained release caffeine.

In some embodiments, instant release caffeine and sustained release caffeine are present in a brain health composition provided herein at a ratio of about 4:1, about 3:1, about 2:1, about 1:1, about 1:2, about 1:3, or about 1:4 instant release caffeine to sustained release caffeine.

### Theacrine

In some embodiments, a brain health composition provided herein includes theacrine. Theacrine is an alkaloid found primarily in Kucha tea. Theacrine may raise dopamine levels and preventing its reabsorption to boost energy and mood. In some embodiments, theacrine can enhance endurance and performance by providing stimulatory effects without adaptation or habituation, and/or reduce stress and anxiety by promoting positive mood elevation and mental clarity, and/or facilitate fat burning by inducing thermogenesis, and/or reducing inflammation throughout the body, and/or act as an antioxidant and protect the cells from damage. Theacrine may also reduce the crash effect of caffeine (e.g., instant release caffeine) when combined with methylliberine.

Any appropriate method can be used to obtain theacrine for use in the brain health compositions provided herein. For example, theacrine can be obtained by chemical synthesis and/or by isolation from tea leaves such as Camellia assamica var. kucha leaves.

The brain health compositions provided herein can contain any appropriate amount of theacrine. In some embodiments, theacrine is present in an amount of at least about 1 mg per serving of a brain health composition provided herein (e.g., at least about 5 mg, at least about 10 mg, at least about 25 mg, at least about 50 mg, at least about 75 mg, at least about 100 mg, at least about 125 mg, at least about 150 mg, or at least about 200 mg per serving of a brain health composition provided herein). In some embodiments, theacrine is present in an amount of no more than about 225 mg per serving of a brain health composition provided herein (e.g., no more than about 200 mg, no more than about 175 mg, no more than about 150 mg, no more than about 125 mg, no more than about 100 mg, no more than about 75 mg, or no more than about 50 mg per serving of a brain health composition provided herein).

In some embodiments, theacrine is present in an amount of about 5 mg to about 250 mg per serving of a brain health composition provided herein. For example, theacrine can be present in an amount of about 5 mg to about 25 mg, about 5 mg to about 50 mg, about 5 mg to about 75 mg, about 5 mg to about 100 mg, about 5 mg to about 125 mg, about 5 mg to about 150 mg, about 5 mg to about 175 mg, about 5 mg to about 200 mg, about 5 mg to about 225 mg, about 25 mg to about 50 mg, about 25 mg to about 75 mg, about 25 mg to about 100 mg, about 25 mg to about 125 mg, about 25 mg to about 150 mg, about 25 mg to about 175 mg, about 25 mg to about 200 mg, about 25 mg to about 225 mg, about 25 mg to about 250 mg, about 50 mg to about 75 mg, about 50 mg to about 100 mg, about 50 mg to about 125 mg, about 50 mg to about 150 mg, about 50 mg to about 175 mg, about 50 mg to about 200 mg, about 50 mg to about 225 mg, about 50 mg to about 250 mg, about 75 mg to about 100 mg, about 75 mg to about 125 mg, about 75 mg to about 150 mg, about 75 mg to about 175 mg, about 75 mg to about 200 mg, about 75 mg to about 225 mg, about 75 mg to about 250 mg, about 100 mg to about 125 mg, about 100 mg to about 150 mg, about 100 mg to about 175 mg, about 100 mg to about 200 mg, about 100 mg to about 225 mg, about 100 mg to about 250 mg, about 125 mg to about 150 mg, about 125 mg to about 175 mg, about 125 mg to about 200 mg, about 125 mg to about 225 mg, about 125 mg to about 250 mg, about 150 mg to about 175 mg, about 150 mg to about 200 mg, about 150 mg to about 225 mg, about 150 mg to about 250 mg, about 175 mg to about 200 mg, about 175 mg to about 225 mg, about 175 mg to about 250 mg, about 200 mg to about 225 mg, about 200 mg to about 250 mg, or about 225 mg to about 250 mg per serving of a brain health composition provided herein. In some embodiments, theacrine is present in an amount of about 40 to about 100 mg per serving of a brain health composition provided herein. In some embodiments, theacrine is present in an amount of about 40 to about 80 mg per serving of a brain health composition provided herein. In some embodiments, theacrine is present in an amount of about 40 to about 60 mg per serving of a brain health composition provided herein. In some embodiments, theacrine is present in an amount of about 60 to about 100 mg per serving of a brain health composition provided herein. In some embodiments, theacrine is present in an amount of about 60 to about 80 mg per serving of a brain health composition provided herein.

In some embodiments, theacrine is present in an amount of about 5 mg, about 20 mg, about 30 mg, about 40 mg, about 50 mg, about 60 mg, about 70 mg, about 80 mg, about 90 mg, about 100 mg, about 150 mg, about 200 mg, or about 250 mg per serving of a brain health composition provided herein.

In some embodiments, theacrine is present in an amount of about 1% to about 40% w/w of a brain health composition provided herein. For example, theacrine can be present in an amount of about 1% to about 5%, about 1% to about 10%, about 1% to about 15%, about 1% to about 20%, about 1% to about 25%, about 1% to about 30%, about 1% to about 35%, about 5% to about 10%, about 5% to about 15%, about 5% to about 20%, about 5% to about 25%, about 5% to about 30%, about 5% to about 35%, about 5% to about 40%, about 10% to about 15%, about 10% to about 20%, about 10% to about 25%, about 10% to about 30%, about 10% to about 35%, about 10% to about 40%, about 15% to about 20%, about 15% to about 25%, about 15% to about 30%, about 15% to about 35%, about 15% to about 40%, about 20% to about 25%, about 20% to about 30%, about 20% to about 35%, about 20% to about 40%, about 25% to about 30%, about 25% to about 35%, about 25% to about 40%, about 30% to about 35%, about 30% to about 40%, or about 30% to about 40% w/w of a brain health composition provided herein. In some embodiments, theacrine is present in an amount of about 5% to about 20% w/w of a brain health composition provided herein. In some embodiments, theacrine is present in an amount of about 1%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 35% or about 40% w/w of a brain health composition provided herein. In some embodiments, theacrine is present in an amount of about 10% w/w of a brain health composition provided herein.

### Methylliberine

In some embodiments, a brain health composition provided herein includes methylliberine. Methylliberine can be found naturally in certain coffee species including Coffea arabica. Methylliberine may activate neurotransmitters such as dopamine receptors. Methylliberine can be helpful for increasing motivation, boosting energy, and/or increasing focus.

Any appropriate method can be used to obtain methylliberine for use in the brain health compositions provided herein. For example, methylliberine can be obtained by chemical synthesis and/or by isolation from tea leaves such as Camellia assamica var. kucha leaves.

The brain health compositions provided herein can contain any appropriate amount of methylliberine. In some embodiments, methylliberine is present in an amount of at least about 1 mg per serving of a brain health composition provided herein (e.g., at least about 5 mg, at least about 10 mg, at least about 20 mg, at least about 50 mg, at least about 75 mg, at least about 100 mg, at least about 125 mg, at least about 150 mg, or at least about 200 mg per serving of a brain health composition provided herein). In some embodiments, methylliberine is present in an amount of no more than about 275 mg per serving of a brain health composition provided herein (e.g., no more than about 250 mg, no more than about 200 mg, no more than about 175 mg, no more than about 150 mg, no more than about 125 mg, no more than about 100 mg, no more than about 75 mg, or no more than about 50 mg per serving of a brain health composition provided herein).

In some embodiments, methylliberine is present in an amount of about 5 mg to about 250 mg per serving of a brain health composition provided herein. For example, methylliberine can be present in an amount of about 5 mg to about 25 mg, about 5 mg to about 50 mg, about 5 mg to about 75 mg, about 5 mg to about 100 mg, about 5 mg to about 125 mg, about 5 mg to about 150 mg, about 5 mg to about 175 mg, about 5 mg to about 200 mg, about 5 mg to about 225 mg, about 25 mg to about 50 mg, about 25 mg to about 75 mg, about 25 mg to about 100 mg, about 25 mg to about 125 mg, about 25 mg to about 150 mg, about 25 mg to about 175 mg, about 25 mg to about 200 mg, about 25 mg to about 225 mg, about 25 mg to about 250 mg, about 50 mg to about 75 mg, about 50 mg to about 100 mg, about 50 mg to about 125 mg, about 50 mg to about 150 mg, about 50 mg to about 175 mg, about 50 mg to about 200 mg, about 50 mg to about 225 mg, about 50 mg to about 250 mg, about 75 mg to about 100 mg, about 75 mg to about 125 mg, about 75 mg to about 150 mg, about 75 mg to about 175 mg, about 75 mg to about 200 mg, about 75 mg to about 225 mg, about 75 mg to about 250 mg, about 100 mg to about 125 mg, about 100 mg to about 150 mg, about 100 mg to about 175 mg, about 100 mg to about 200 mg, about 100 mg to about 225 mg, about 100 mg to about 250 mg, about 125 mg to about 150 mg, about 125 mg to about 175 mg, about 125 mg to about 200 mg, about 125 mg to about 225 mg, about 125 mg to about 250 mg, about 150 mg to about 175 mg, about 150 mg to about 200 mg, about 150 mg to about 225 mg, about 150 mg to about 250 mg, about 175 mg to about 200 mg, about 175 mg to about 225 mg, about 175 mg to about 250 mg, about 200 mg to about 225 mg, about 200 mg to about 250 mg, or about 225 mg to about 250 mg per serving of a brain health composition provided herein. In some embodiments, methylliberine is present in an amount of about 40 mg to about 100 mg per serving of a brain health composition provided herein. In some embodiments, methylliberine is present in an amount of about 50 mg to about 100 mg per serving of a brain health composition provided herein. In some embodiments, methylliberine is present in an amount of about 80 mg to about 100 mg per serving of a brain health composition provided herein. In some embodiments, methylliberine is present in an amount of about 70 mg to about 80 mg per serving of a brain health composition provided herein. In some embodiments, methylliberine is present in an amount of about 50 mg to about 70 mg per serving of a brain health composition provided herein.

In some embodiments, methylliberine is present in an amount of about 5 mg, about 25 mg, about 55 mg, about 65 mg, about 75 mg, about 85 mg, about 95 mg, about 105 mg, about 125 mg, about 175 mg, or about 250 mg per serving of a brain health composition provided herein.

In some embodiments, methylliberine is present in an amount of about 3% to about 50% w/w of a brain health composition provided herein. For example, methylliberine can be present in an amount of about 3% to about 5%, about 3% to about 10%, about 3% to about 15%, about 3% to about 20%, about 3% to about 25%, about 3% to about 30%, about 3% to about 35%, about 3% to about 40%, about 3% to about 45%, about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 10% to about 15%, about 10% to about 20%, about 10% to about 25%, about 10% to about 30%, about 10% to about 35%, about 10% to about 40%, about 10% to about 45%, about 10% to about 50%, about 15% to about 20%, about 15% to about 25%, about 15% to about 30%, about 15% to about 35%, about 15% to about 40%, about 15% to about 45%, about 15% to about 50%, about 20% to about 25%, about 20% to about 30%, about 20% to about 35%, about 20% to about 40%, about 20% to about 45%, about 20% to about 50%, about 25% to about 30%, about 25% to about 35%, about 25% to about 40%, about 25% to about 45%, about 25% to about 50%, about 30% to about 35%, about 30% to about 40%, about 30% to about 45%, about 30% to about 50%, about 30% to about 40%, about 30% to about 45%, about 30% to about 50%, about 40% to about 45%, about 40% to about 50%, or about 45% to about 50% w/w of a brain health composition provided herein. In some embodiments, methylliberine is present in an amount of about 8% to about 25% w/w of a brain health composition provided herein. In some embodiments, methylliberine is present in an amount of about 3%, about 5%, about 10%, about 11%, about 12%, about 13%, about 14%, about 15%, about 16%, about 17%, about 18%, about 19%, about 20%, about 21%, about 22%, about 23%, about 24%, about 25%, about 30%, about 35%, about 40%, about 45%, or about 50% w/w of a brain health composition provided herein. In some embodiments, methylliberine is present in an amount of about 15% w/w of a brain health composition provided herein.

### Theanine

The brain health compositions provided herein include theanine. Theanine can be found in green tea. Theanine can help promote relaxation without sedation. This effect can help “take the edge off” of caffeine and can result in synergistic effects. For example, the combination of theanine and caffeine can outperform caffeine alone on measures of one or more of accuracy, attention, sustained, attention, reaction time, alertness, task switching, and fatigue.

Any appropriate method can be used to obtain theanine for use in the brain health compositions provided herein. For example, theanine can be obtained by chemical synthesis, enzymatic transformation, or plant extraction. In some embodiments, the theanine is from a tea extract. In some embodiments, the theanine is from an extract of green tea. In some embodiments, the theanine is L-theanine.

The brain health compositions provided herein can contain any appropriate amount of theanine. In some embodiments, theanine is present in an amount of at least about 50 mg per serving of a brain health composition provided herein (e.g., at least about 75 mg, at least about 100 mg, at least about 125 mg, at least about 150 mg, at least about 175 mg, at least about 200 mg, at least about 225 mg, at least about 250 mg, or at least 275 mg per serving of a brain health composition provided herein). In some embodiments, theanine is present in an amount of no more than about 300 mg per serving of a brain health composition provided herein (e.g., no more than about 275 mg, no more than about 250 mg, no more than about 225 mg, no more than about 200 mg, no more than about 175 mg, no more than about 150 mg, no more than about 125 mg, or no more than about 100 mg per serving of a brain health composition provided herein).

In some embodiments, theanine is present in an amount of about 50 mg to about 300 mg per serving of a brain health composition provided herein. For example, theanine can be present in an amount of about 50 mg to about 75 mg, about 50 mg to about 100 mg, about 50 mg to about 125 mg, about 50 mg to about 175 mg, about 50 mg to about 200 mg, about 50 mg to about 225 mg, about 50 mg to about 250 mg, about 50 mg to about 275 mg, about 75 mg to about 100 mg, about 75 mg to about 125 mg, about 75 mg to about 150 mg, about 75 mg to about 175 mg, about 75 mg to about 200 mg, about 75 mg to about 225 mg, about 75 mg to about 250 mg, about 75 mg to about 275 mg, about 75 mg to about 300 mg, about 100 mg to about 125 mg, about 100 mg to about 150 mg, about 100 mg to about 175 mg, about 100 mg to about 200 mg, about 100 mg to about 225 mg, about 100 mg to about 250 mg, about 100 mg to about 275 mg, about 100 mg to about 300 mg, about 125 mg to about 150 mg, about 125 mg to about 175 mg, about 125 mg to about 200 mg, about 125 mg to about 225 mg, about 125 mg to about 250 mg, about 125 mg to about 275 mg, about 125 mg to about 300 mg, about 150 mg to about 175 mg, about 150 mg to about 200 mg, about 150 mg to about 225 mg, about 150 mg to about 250 mg, about 150 mg to about 275 mg, about 150 mg to about 300 mg, about 175 mg to about 200 mg, about 175 mg to about 225 mg, about 175 mg to about 250 mg, about 175 mg to about 275 mg, about 175 mg to about 300 mg, about 200 mg to about 225 mg, about 200 mg to about 250 mg, about 200 mg to about 275 mg, about 200 mg to about 300 mg, about 225 mg to about 250 mg, about 225 mg to about 275 mg, about 225 mg to about 300 mg, about 250 mg to about 275 mg, about 250 mg to about 300 mg, or about 275 mg to about 300 mg per serving of a brain health composition provided herein. In some embodiments, theanine is present in an amount of about 100 mg to about 200 mg per serving of a brain health composition provided herein. In some embodiments, theanine is present in an amount of about 50 mg, about 100 mg, about 125 mg, about 150 mg, about 175 mg, about 200 mg, about 250 mg, about 300 mg per serving of a brain health composition provided herein.

In some embodiments, theanine is present in an amount of about 10% to about 60% w/w of a brain health composition provided herein. For example, theanine can be present in an amount of about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 20% to about 25%, about 20% to about 30%, about 20% to about 35%, about 20% to about 40%, about 20% to about 45%, about 20% to about 50%, about 20% to about 55%, about 20% to about 60%, about 25% to about 30%, about 25% to about 35%, about 25% to about 40%, about 25% to about 45%, about 25% to about 50%, about 25% to about 55%, about 25% to about 60%, about 30% to about 35%, about 30% to about 40%, about 30% to about 45%, about 30% to about 50%, about 30% to about 55%, about 30% to about 60%, about 35% to about 40%, about 35% to about 45%, about 35% to about 50%, about 35% to about 55%, about 35% to about 60%, about 40% to about 45%, about 40% to about 50%, about 40% to about 55%, about 40% to about 60%, about 45% to about 50%, about 45% to about 55%, about 45% to about 60%, about 50% to about 55%, about 50% to about 60%, or about 55% to about 60% w/w of a brain health composition provided herein. In some embodiments, theanine is present in an amount of about 20% to about 40% w/w of a brain health composition provided herein. In some embodiments, theanine is present in an amount of about 10%, about 15%, about 20%, about 25%, about 30%, about 35%, about 40%, about 45%, about 50%, about 55%, or about 60% w/w of a brain health composition provided herein.

In some embodiments, a brain health composition provided herein is a composition that includes the ingredients listed in Table 1.

TABLE 1

| | Amount |
|---|---|
| Ingredient | (mg per serving) |
| Caffeine (e.g., anhydrous and/or | 60 to 200 |
| instant release) | |
| Sustained release Caffeine | 50 to 200 |
| Methylliberine | 40 to 100 |
| Theacrine | 40 to 100 |
| Coffeeberry extract | 50 to 250 |

In some embodiments, a brain health composition provided herein is a composition that includes the ingredients listed in Table 2.

TABLE 2

| | Amount |
|---|---|
| Ingredient | (mg per serving) |
| Caffeine (e.g., instant release and/or | 180 |
| sustained release | |
| Blend of Methylliberine and Theacrine | 125 |
| Coffeeberry extract | 100 |

In some embodiments, a brain health composition provided herein is a composition that includes the ingredients listed in Table 3.

TABLE 3

| | Amount |
|---|---|
| Ingredient | (mg per serving) |
| Caffeine (e.g., anhydrous and/or | 60 to 200 |
| instant release) | |
| Sustained release Caffeine | 50 to 200 |
| L-Theanine | 60 to 200 |
| Coffeeberry extract | 50 to 250 |

In some embodiments, a brain health composition provided herein is a composition that includes the ingredients listed in Table 4.

TABLE 4

| | Amount |
|---|---|
| Ingredient | (mg per serving) |
| Caffeine (e.g., instant release and/or | 180 |
| sustained release | |
| L-Theanine | 200 |
| Coffeeberry extract | 100 |

A brain health composition provided herein can be formulated for oral use and can further include one or more suitable excipients, sweetening agents, flavorants, colorants, preservatives, and/or other ingredients. Non-limiting examples of oral formulations include tablets, troches, lozenges, aqueous or oil suspensions, non-aqueous solutions, dispersible powders or granules (including micronized particles or nanoparticles), emulsions, hard or soft capsules, syrups, and elixirs. Compositions intended for oral use can be prepared according to any appropriate method, including standard methods for the manufacture of pharmaceutical compositions. Preparations for oral use can be suitably formulated to give controlled or sustained release of one or more compounds.

Liquid preparations for oral administration can take the form of, for example, solutions, syrups, or suspension, or they can be presented as a dry product for reconstitution with saline or other suitable liquid vehicle before use. In some embodiments, liquid preparations can contain pharmaceutically acceptable additives such as suspending agents, emulsifying agents, non-aqueous vehicles, preservatives, buffer salts, flavoring agents, coloring agents, and sweetening agents as appropriate.

Tablets, capsules, or soft gels can be prepared with one or more suitable excipients, such as binding agents, fillers, lubricants, disintegrants, and/or wetting agents. In some embodiments, a capsule, tablet, or soft gel form of the brain health compositions provided herein further include one or more of microcrystalline cellulose, hydroxypropyl methylcellulose, silicon dioxide, and magnesium stearate.

In some embodiments, a tablet of a brain health composition provided herein can have a coating (e.g., a polymer or polysaccharide-based coating with or without plasticizers and/or pigments). Such coatings can include, for example, one or more of a film-forming polymer, a colorant, and a flavorant. Non-limiting examples of a flavorant include vanillin, vanilla extract, artificial vanilla flavorant, and natural caramel. In some embodiments, a brain health composition can have a vanilla or vanilla caramel flavor profile. In some embodiments, a coating of a tablet of a brain health composition provided herein includes one or more of: hydroxypropyl methyl cellulose, sodium alginate, calcium carbonate, calcium sulfate, magnesium carbonate, cellulose gum, isomalt, polyethylene glycol, glycerin, hydroxypropyl cellulose, and carnauba wax.

In some embodiments, a capsule, tablet, or soft gel form of the brain health compositions provided herein can have a unit dosage equal to the daily desired dosage for a particular mammal. For example, if it is desired to administer 100 mg of a particular agent to a mammal, each tablet can include about 100 mg in weight of that agent. As used herein, mammals generally refer to humans, but also can include domesticated mammals (e.g., dogs, cats, and livestock such as cows, horses, pigs, or sheep). The dosages of a particular brain health composition provided herein will depend on many factors, including the general health of a mammal. In some cases, a total daily dose (e.g., a full serving) can be prepared and administered in the form of one or more dosage units (e.g., one tablet, capsule, or soft gel, two tablets, capsules, or soft gels, three tablets, capsules, or soft gels, four tablets, capsules or soft gels, five tablets, capsules, or soft gels, or six tablets, capsules, or soft gels). For instance, in some cases, one tablet, one capsule, or one soft gel can include a full serving (e.g., daily dosage) of the composition. In some cases, two tablets, two capsules, or two soft gels in combination can include a full serving (e.g., daily dosage) of the composition. In some cases, three tablets, three capsules, or three soft gels in combination can include a full serving (e.g., daily dosage) of the composition.

In some embodiments, a brain health composition provided herein can be substantially free of artificial colors, artificial flavors, and chemical preservatives.

In some embodiments, a brain health composition provided herein is a composition that includes the ingredients listed in Table 5.

TABLE 5

| | Amount | |
|---|---|---|
| | (mg per | Amount |
| Ingredient | serving) | (% w/w) |
| Caffeine (e.g., anhydrous and/or instant release) | 60 to 200 | 15 to 40 |
| Sustained release Caffeine | 50 to 200 | 5 to 15 |
| Methylliberine | 40 to 100 | 8 to 25 |
| Theacrine | 40 to 100 | 5 to 20 |
| Coffeeberry extract | 50 to 250 | 10 to 30 |
| Binding agent(s), filler(s), lubricant(s), | 25 to 100 | 1 to 40 |
| disintegrant(s), colorant(s), and/or flavorant(s) | | |

In some embodiments, a brain health composition provided herein is a composition that includes the ingredients listed in Table 6.

TABLE 6

| | Amount | |
|---|---|---|
| | (mg per | Amount |
| Ingredient | serving) | (% w/w) |
| Caffeine (e.g., instant release and/or sustained | 180 | 44.5 |
| release) | | |
| Blend of Methylliberine and Theacrine | 125 | 25 |
| Coffeeberry extract | 100 | 20 |
| Binding agent(s), filler(s), lubricant(s), | 37.2 | 10.5 |
| disintegrant(s), colorant(s), and/or flavorant(s) | | |

### Methods

The brain health compositions provided herein can be useful for one or more of reducing fatigue; supporting healthy energy levels; increasing vigor; improving alertness; reducing anxiety; improving reaction time; improving attention; improving mental accuracy; improving working memory; increasing vigilance; reducing test errors; improving video game performance; increasing levels of BDNF in the brain; increasing brain activity; supporting mood; and supporting brain function.

Provided herein are methods of improving one or more of alertness, reaction time, attention, mental accuracy, working memory, and video game performance in a mammal, comprising administering to the mammal any of the brain health compositions described herein. In some embodiments, the method comprises improving attention and/or alertness in the mammal (e.g., after administration of a brain health composition described herein as compared to before administration of a brain health composition described herein). In some embodiments, the method comprises improving reaction time and/or video game performance in the mammal (e.g., after administration of a brain health composition described herein as compared to before administration of a brain health composition described herein). In some embodiments, the method comprises improving mental accuracy and/or working memory in the mammal (e.g., after administration of a brain health composition described herein as compared to before administration of a brain health composition described herein).

Also provided herein are methods of reducing one or more of fatigue, anxiety, occasional feelings of stress, and test errors in a mammal, comprising administering to the mammal any of the brain health compositions described herein. In some embodiments, the method comprises reducing fatigue in the mammal (e.g., after administration of a brain health composition described herein as compared to before administration of a brain health composition described herein). In some embodiments, the method comprises reducing anxiety in the mammal (e.g., after administration of a brain health composition described herein as compared to before administration of a brain health composition described herein). In some embodiments, the method comprises reducing occasional feelings of stress in the mammal (e.g., after administration of a brain health composition described herein as compared to before administration of a brain health composition described herein). In some embodiments, the method comprises reducing test errors in the mammal (e.g., after administration of a brain health composition described herein as compared to before administration of a brain health composition described herein).

Also provided herein are methods of supporting one or more of healthy energy levels, mood, and brain function in a mammal, comprising administering to the mammal any of the brain health compositions described herein. In some embodiments, the method comprises supporting healthy energy levels in the mammal (e.g., after administration of a brain health composition described herein as compared to before administration of a brain health composition described herein). In some embodiments, the method comprises supporting mood in the mammal (e.g., after administration of a brain health composition described herein as compared to before administration of a brain health composition described herein). In some embodiments, the method comprises supporting brain function in the mammal (e.g., after administration of a brain health composition described herein as compared to before administration of a brain health composition described herein).

Also provided herein are methods of increasing one or more of vigor, vigilance, levels of BDNF in the brain, and brain activity in a mammal, comprising administering to the mammal any of the brain health compositions described herein. In some embodiments, the method comprises increasing vigor in the mammal (e.g., after administration of a brain health composition described herein as compared to before administration of a brain health composition described herein). In some embodiments, the method comprises increasing vigilance in the mammal (e.g., after administration of a brain health composition described herein as compared to before administration of a brain health composition described herein). In some embodiments, the method comprises increasing level of BDNF in the brain of the mammal (e.g., after administration of a brain health composition described herein as compared to before administration of a brain health composition described herein). In some embodiments, the method comprises increasing brain activity in the mammal (e.g., after administration of a brain health composition described herein as compared to before administration of a brain health composition described herein).

In some embodiments, a mammal does not exhibit jittery feelings after administration of a brain health composition described herein (e.g., as compared to before administration of a brain health composition described herein).

In some embodiments, a mammal does not exhibit a caffeine crash after administration of a brain health composition described herein (e.g., as compared to administration of instant release caffeine alone).

In some embodiments, the combination of caffeine (e.g., instant release caffeine and/or sustained release caffeine), theacrine, methylliberine, and a coffeeberry extract improve one or more of focus, energy, mood, attention, and performance (e.g., as compared to any one of caffeine (e.g., instant release caffeine and/or sustained release caffeine), theacrine, methylliberine, and a coffeeberry extract consumed alone).

In some embodiments, the combination of caffeine (e.g., instant release caffeine and/or sustained release caffeine), theanine, and a coffeeberry extract improve one or more of focus, energy, mood, attention, and performance (e.g., as compared to any one of caffeine (e.g., instant release caffeine and/or sustained release caffeine), theanine, and a coffeeberry extract consumed alone).

Fatigue, energy levels, vigor, alertness, anxiety, reaction time, attention, mental accuracy, working memory, vigilance, the number of test errors, video game performance, levels of BDNF in the brain, brain activity, mood, and brain function can each be assessed using any appropriate method(s). Non-limiting examples of methods that can be used to assess one or more of fatigue, energy levels, vigor, alertness, anxiety, reaction time, attention, mental accuracy, working memory, vigilance, the number of test errors, video game performance, and mood include Profile of Mood States (POMS) survey, Mental Fatigue, Alertness, and Motivation Visual Analogue Scales (VAS), Mental and Physical State Energy and Fatigue Scales, Bond-Lader Scale, Delayed Word Recall, Delayed Word Recognition, Psychomotor Vigilance Test, Flanker Inhibitory Control and Attention Test, Pattern Comparison Processing Speed Test, Dimensional Change Card Sort Test, and Mood, Alertness, and Physical Sensations Scales (MAPPS). See, e.g., Reed et al. J. Cogn. Enhanc. 3:193-206 (2019); Tartar et al. Cureus 13 (2021).

POMS is a self-report questionnaire that is widely used to measure mood. See e.g., Morfeld et al. Psychosoc. Med. 4: Doc06 (2007). Mental Fatigue, Alertness, and Motivation Visual Analogue Scales can be used to assess current feelings of mental fatigue, alertness, and motivation. For example, the mental fatigue and alertness scales can be scored from “not at all” to “extremely,” while the motivation scale can be scored from “no motivation at all” to “strongest degree of motivation imaginable,” with scores ranging from 0 to 100. Mental and Physical State Energy and Fatigue Scales (EFS-State Scales) can include reporting of a mammal's (e.g., a human) current subjective feelings of physical energy, physical fatigue, mental energy, and mental fatigue. In some embodiments, current subjective feelings can be reported by selecting a point along a visual analog scale for three items for each EFS-State scale dimension. For example, energy items can include (i) “I feel I have no energy” to “strongest feelings of energy ever felt”; (ii) “I feel I have no vigor” to “strongest feelings of vigor ever felt”; and (iii) “I feel I have no pep” to “strongest feelings of pep ever felt”; fatigue items can include (i) “I feel no fatigue” to “strongest feelings of fatigue ever felt”; (ii) “I feel no exhaustion” to “strongest feelings of exhaustion ever felt”; and (iii) “I have no feelings of being worn out” to “strongest feelings of being worn out ever felt.” The Bond-Lader Scale is derived from 16 scales that are anchored on each side by adjectives describing a mood and can provide three composite scores for Alert, Calm, and Content. See, e.g., Reed et al. J. Cogn. Enhanc. 3:193-206 (2019).

Non-limiting examples of methods that can be used to assess one or more of levels of BDNF in the brain, brain activity, mood, and brain function include physiological measures of stress and arousal such as measurements of neurophysiological arousal, measurements of biomarkers of arousal, and measurements of heart rate and blood pressure. For example, an electroencephalogram (EEG), salivary cortisol, and alpha-amylase (sAA) can be used to assess stress and arousal. See, e.g., Tartar et al. Cureus 13 (2021).

The frequency of consuming a brain health composition provided herein can vary from once or twice a week to once or more times a day, such as two or three times daily. In some embodiments, a brain health composition provided herein is consumed once daily. The frequency of consuming a brain health composition provided herein can be constant or be variable during the duration of the treatment. In some embodiments, about one serving of a brain health composition provided herein is consumed per day.

In some embodiments, a total daily dose (e.g., a full serving) of a brain health composition provided herein is consumed in the form of one dosage units (e.g., one tablet, capsule, or soft gel).

In some embodiments, a brain health composition provided herein is consumed by a mammal about 6 hours to about 15 hours prior to a desirable time to fall asleep (e.g., bedtime). In some embodiments, a brain health composition provided herein is consumed by a mammal about 8 hours to about 12 hours prior to a desirable time to fall asleep (e.g., bedtime). In some embodiments, a brain health composition provided herein is consumed at least 8 hours prior to a desirable time to fall asleep (e.g., bedtime).

### EXAMPLES

### Example 1. Exemplary Brain Health Compositions

A composition as shown in Table 7 was prepared. This composition is also referred to herein as Composition A.

TABLE 7
Composition A.

| | Amount (per | | Amount |
|---|---|---|---|
| Ingredient | serving) | | (% w/w) |
| Caffeine (instant release and sustained | 180 | mg | 43 |
| release) | | | |
| Blend of methylliberine and theacrine | 125 | mg | 30 |
| Coffeeberry extract | 100 | mg | 24 |
| Binding agent(s), filler(s), lubricant(s), | 15.1 | mg | 3 |
| disintegrant(s), colorant(s), and/or | | | |
| flavorant(s) | | | |

A composition as shown in Table 8 was prepared.

TABLE 8

| | Amount | Amount |
|---|---|---|
| Ingredient | (per serving) | (% w/w) |
| Caffeine (instant release and sustained release) | 180 mg | 43 |
| L-Theanine | 200 mg | 38 |
| Coffeeberry extract | 100 mg | 19 |

A composition as shown in Table 9 was prepared. This composition is also referred to herein as Composition B.

TABLE 9
Composition B.

| | Amount |
|---|---|
| Ingredient | (per serving) |
| Caffeine (instant release and sustained release) | 180 mg |
| Blend of theanine and theacrine | 125 mg |
| Coffeeberry extract | 100 mg |

### Example 2. Pilot Study

### Methods

Twelve different ingredients or formulations were evaluated. Subjective tests included a survey using a 5-point Likert scale with questions about, e.g., whether the participant felt the product working and the timing of when the participant felt the product working. The survey asked the participants to evaluate a statement by giving it a quantitative value based on level of agreement/disagreement, e.g., where 1 is strongly disagree; 2 is disagree; 3 is neither agree nor disagree; 4 is agree; and 5 is strongly agree.

The ingredient and formulations were also evaluated for their anecdotal effects on same-day cognition, energy, and mood compared to an inert placebo. Participants were randomly provided 1 serving in the early afternoon (about 1 pm) and asked to submit their survey response at the end of their workday. All compositions in Table 9 were administered as capsules.

### Results

Of Samples 1-4, Sample 3 scored the highest on a 5-point Likert scale for whether participants felt the product working. See Table 10.

TABLE 10

| | | | Sample 3: | |
|---|---|---|---|---|
| | | | IR Caffeine + | |
| | Sample 1: | | SR Caffeine + | Sample 4: |
| | IR Caffeine* + | Sample 2: | Theacrine + | IR Caffeine + |
| Variable | SR Caffeine** | Placebo | Methylliberine | Theanine |
| Felt Product | 4.2 | 3.0 | 4.3 | 3.2 |
| Working | | | | |

*IR caffeine refers to instant release caffeine.
**SR caffeine refers to sustained release caffeine.

All responses for the samples in Table 11 indicated an onset within 1 hour of 5 ingestion.

TABLE 11

| | | | Sample 3: | |
|---|---|---|---|---|
| | | | IR Caffeine + | |
| | Sample 1: | | SR Caffeine + | Sample 4: |
| | IR Caffeine + | Sample 2: | Theacrine + | IR Caffeine + |
| Variable | SR Caffeine | Placebo | Methylliberine | Theanine |
| No Onset | 0% | 25% | 0% | 20% |
| Within 30 Minutes | 60% | 0% | 71% | 40% |
| 31-60 Minutes | 40% | 75% | 29% | 40% |
| Longer than 1 hour | 0% | 0% | 0% | 0% |

The combination of theacrine with methylliberine and caffeine (whether instant release or instant release+sustained release) outperformed all other samples tested using a 5-point Likert scale for focus, energy, attention, alertness, memory, and mood. For example, compare Samples 3 and 3a with Samples 1, 2, and 4. See Table 12.

TABLE 12

| | | | Sample 3: | | |
|---|---|---|---|---|---|
| | | | IR Caffeine + | Sample 3a: | |
| | Sample 1: | | SR Caffeine + | IR Caffeine + | Sample 4: |
| | IR Caffeine + | Sample 2: | Theacrine + | Theacrine + | IR Caffeine + |
| Variable | SR Caffeine | Placebo | Methylliberine | Methylliberine | Theanine |
| Focus | 3.6 | 3.5 | 4.3 | 4.4 | 3.8 |
| Energy | 4.2 | 3.8 | 4.6 | 5.0 | 4.0 |
| Attention | 3.4 | 3.8 | 4.3 | 4.2 | 3.0 |
| Alertness | 4.0 | 4.3 | 4.6 | 5.0 | 3.4 |
| Memory | 3.2 | 3.3 | 3.9 | 4.0 | 3.6 |
| Mood | 3.6 | 3.5 | 4.3 | 4.2 | 2.8 |

### Example 3. Clinical Study

Pilot studies were used to determine ingredient options that demonstrated an acute improvement in energy or cognition subjectively. These data were used to formulate Composition A.

### Methods

The Low-Caffeine User Clinical Study was randomized, single-blind, within-subjects, and acute design. This study included subjective (POMS and product questions) and objective (CNSVS) tests. The treatments were as follows: placebo (500 mg rice flour); caffeine (180 mg caffeine anhydrous+320 mg rice flour); Composition A; and Composition B. The Profile of Mood States (POMS) survey measured several mood domains including fatigue and vigor (energy) together with tension, anger, depression, self-esteem, and confusion to aggregates as total mood disturbance (TMD).

Thirty out of thirty-two participants completed the trial. The participants averaged 93 mg average caffeine intake per day. There were 13 male and 17 female participants with an average age of 37.1 years. The participants were healthy by self-report with no known intolerances to caffeine at baseline, no personal or immediate family history of attention or mood disorder, and no usage of any competing medication or supplements.

The Moderate-Caffeine User Follow-Up was a single-blind, acute design. The study included subjective tests (POMS and product questions) only. The treatments were as follows: placebo (500 mg rice flour) randomized with caffeine; caffeine (180 mg caffeine anhydrous+320 mg rice flour) randomized with placebo; Composition A; and Composition B.

Thirteen participants completed the trial (1 completed ¾ arms due to scheduling interference). The participants averaged 338 mg caffeine intake per day. There were 7 male and 6 female participants with an average age of 39.5 years. The participants were healthy by self-report, with no known intolerances to caffeine at baseline, no personal or immediate family history of attention or mood disorder, and no usage of any competing medication or supplements.

### Results

Low-Caffeine User Clinical Trial. Objective tests were only performed on Composition A and placebo. Using RMANOVA, no significant interactions were observed. Using T-tests on the deltas, SAT correct responses significantly improved with Composition A vs. placebo. Composition A improved several cognitive domains while Placebo was unchanged (within group). See Table 13.

TABLE 13

| Variable | Composition A | | | Placebo | | |
|---|---|---|---|---|---|---|
| (n = 30) | Pre | Post | Delta | Pre | Post | Delta |
| Processing Speed | 65.9 ± 10.6 | 70.7 ± 10.7 | 4.8 ± 7.8 | 67.9 ± 11.3 | 70.1 ± 10.1 | 2.1 ± 12.5 |
| Patient Score | | | | | | |
| Processing Speed | 108.6 ± 16.6 | 117.2 ± 15.6^{†} | 8.6 ± 15.7 | 112.5 ± 14.8 | 116.5 ± 14.6 | 4.0 ± 16.8 |
| Standard Score | | | | | | |
| Executive Function | 45.9 ± 20.2 | 57.6 ± 9.38^{†} | 11.7 ± 16.8 | 52.7 ± 10.8 | 56.8 ± 7.5 | 4.1 ± 8.1 |
| patient score | | | | | | |
| Executive Function | 97.7 ± 27.9 | 113.0 ± 12.2^{†} | 15.2 ± 23.9 | 107.0 ± 15.1 | 112.4 ± 11.1 | 5.4 ± 11.3 |
| standard score | | | | | | |
| SDC correct | 66.6 ± 10.9 | 70.3 ± 16.5 | 3.7 ± 12.6 | 69.0 ± 12.8 | 71.7 ± 9.6 | 2.7 ± 12.0 |
| responses | | | | | | |
| SDC correct | 110.0 ± 14.1 | 118.0 ± 15.6 | 8.0 ± 9.9 | 113.0 ± 15.8 | 117.3 ± 13.5 | 4.3 ± 16.3 |
| responses | | | | | | |
| standard score | | | | | | |
| SDC errors | 0.70 ± 1.20 | 1.60 ± 2.7^{†} | 0.9 ± 1.9 | 1.43 ± 3.40 | 1.66 ± 3.12 | 0.2 ± 1.4 |
| SDC errors | 103.7 ± 12.0 | 94.5 ± 26.7 | −9.2 ± 19.2 | 96.2 ± 33.5 | 93.8 ± 30.5 | −2.4 ± 14.8 |
| standard score | | | | | | |
| SAT correct | 52.6 ± 11.9 | 60.5 ± 6.87^{†} | 7.9 ± 9.2* | 57.1 ± 8.21 | 60.2 ± 6.3 | 3.1 ± 5.1 |
| responses | | | | | | |
| SAT correct | 99.6 ± 22.5 | 114.5 ± 3.4^{†} | 14.9 ± 18.4* | 108.1 ± 16.1 | 114.0 ± 12.6 | 5.9 ± 10.0 |
| standard score | | | | | | |
| SAT errors | 6.73 ± 9.25 | 3.40 ± 3.97 | −3.3 ± 8.6 | 4.40 ± 4.12 | 3.40 ± 2.98 | −1.0 ± 3.5 |
| SAT errors | 95.6 ± 29.8 | 106.4 ± 13.0 | 10.8 ± 27.6 | 102.6 ± 13.8 | 106.1 ± 10.1 | 3.53 ± 11.7 |
| standard score | | | | | | |
| SAT correct | 997.3 ± 223.9 | 930.1 ± 144.1 | −67.2 ± 208.0 | 975.7 ± 154.2 | 931.7 ± 134.7 | −44.0 ± 77.9 |
| reaction time (ms) | | | | | | |
| SAT reaction time | 105.4 ± 20.2 | 111.7 ± 13.5 | 6.2 ± 19.8 | 107.1 ± 15.7 | 111.5 ± 12.5 | 4.4 ± 7.3 |
| standard score | | | | | | |

SDC—Symbol Digit Coding, a test of information processing;
SAT—Shifting Attention Test, a test of reaction time and executive function
*p < 0.05 vs placebo (comparison of deltas only);
^{†}p < 0.05 from pre within group

Subjective tests were performed on Composition A, Composition B, Caffeine, and Placebo. Over the two-hour experience with the product, users felt an improvement in fatigue in all conditions. However, only Composition A and Composition B improved vigor (energy) over time. See Table 14.

TABLE 14

| Variable | Caffeine | | Composition A | | Composition B | | Placebo | |
|---|---|---|---|---|---|---|---|---|
| (n = 30) | Pre | Post | Pre | Post | Pre | Post | Pre | Post |
| Tension | 4.48 ± | 2.80 ± | 3.87 ± | 2.87 ± | 3.80 ± | 2.10 ± | 4.53 ± | 2.40 ± |
| | 4.79 | 3.96 | 4.38 | 4.10 | 3.79 | 3.43 | 3.95 | 3.17* |
| Anger | 1.55 ± | 0.90 ± | 1.40 ± | 0.47 ± | 1.00 ± | 0.40 ± | 1.38 ± | 0.80 ± |
| | 2.01 | 1.71 | 3.36 | 1.48 | 1.66 | 0.97 | 2.73 | 1.79 |
| Fatigue | 5.66 ± | 2.77 ± | 5.13 ± | 2.60 ± | 4.27 ± | 1.57 ± | 4.50 ± | 2.60 ± |
| | 4.57 | 3.13* | 4.03 | 4.24* | 4.04 | 2.45* | 3.49 | 2.92* |
| Depression | 1.79 ± | 0.60 ± | 1.60 ± | 1.13 ± | 0.97 ± | 0.33 ± | 1.75 ± | 0.57 ± |
| | 2.99 | 1.25 | 3.08 | 3.88 | 1.27 | 1.30 | 2.94 | 1.14* |
| Esteem- | 13.59 ± | 14.63 ± | 14.67 ± | 15.07 ± | 14.37 ± | 15.30 ± | 13.78 ± | 13.97 ± |
| related Affect | 3.06 | 3.69 | 3.31 | 4.06 | 3.01 | 3.16 | 2.68 | 3.05 |
| Vigor | 5.69 ± | 7.27 ± | 5.40 ± | 7.63 ± | 4.90 ± | 8.07 ± | 5.00 ± | 6.10 ± |
| | 3.54 | 4.40 | 3.18 | 4.33* | 3.16 | 3.72* | 3.24 | 3.50 |
| Confusion | 2.93 ± | 1.20 ± | 2.37 ± | 1.33 ± | 2.23 ± | 0.77 ± | 2.38 ± | 1.40 ± |
| | 3.15 | 1.83* | 2.55 | 2.48 | 1.92 | 1.17* | 2.42 | 1.48 |
| TMD | −2.86 ± | −13.63 ± | −5.70 ± | −14.30 ± | −7.00 ± | −18.20 ± | −4.25 ± | −12.30 ± |
| | 14.61 | 11.74 | 16.16 | 18.36 | 11.07 | 8.24* | 12.12 | 8.74* |

*p < 0.05 different from Pre within group

Moderate-Caffeine User Follow-Up. Among those with moderate caffeine intake, the caffeine and Composition A arms performed better. Composition A was significantly better than placebo for enhancing attention. See Table 15.

TABLE 15

| | Moderate-Dose Caffeine Cohort (mean 338 mg/d) | | | | | | | |
|---|---|---|---|---|---|---|---|---|
| | % Top 2 Box | | | | Scores | | | |
| Variable | Caffeine | Comp. A | Comp. B | Placebo | Caffeine | Comp. A | Comp. B | Placebo |
| (n = 12-13) | Statement Agreement | | | | | | | |
| I was better | 58% | 85% | 38% | 54% | 3.25 ± | 3.92 ± | 3.15 ± | 3.00 ± |
| able to focus | | | | | 1.00 | 0.49 | 1.28 | 1.29 |
| after taking | | | | | | | | |
| the product | | | | | | | | |
| The product | 67% | 92% | 38% | 62% | 3.75 ± | 4.23 ± | 3.08 ± | 3.08 ± |
| improved my | | | | | 1.29 | 0.60 | 1.32 | 1.50 |
| energy levels** | | | | | | | | |
| I was more | 58% | 85% | 46% | 54% | 3.67 ± | 4.23 ± | 3.23 ± | 3.00 ± |
| alert after | | | | | 1.15 | 0.73 | 1.42 | 1.29 |
| taking the | | | | | | | | |
| product** | | | | | | | | |
| The product | 58% | 85% | 54% | 38% | 3.50 ± | 4.08 ± | 3.38 ± | 2.77 ± |
| enhanced my | | | | | 1.00 | 0.64^{†} | 1.39 | 1.30 |
| ability to pay | | | | | | | | |
| attention* | | | | | | | | |
| I had no trouble | 58% | 85% | 54% | 46% | 3.67 ± | 4.00 ± | 3.54 ± | 3.23 ± |
| recalling | | | | | 1.15 | 0.58 | 1.33 | 1.48 |
| [things] after | | | | | | | | |
| taking the | | | | | | | | |
| product | | | | | | | | |
| I am in a better | 42% | 85% | 46% | 54% | 3.33 ± | 3.92 ± | 3.38 ± | 3.15 ± |
| mood after | | | | | 1.15 | 0.49 | 1.12 | 1.34 |
| having taken | | | | | | | | |
| the product | | | | | | | | |

*p < 0.05 one-way ANOVA;
**Trend p < 0.1 one-way ANOVA;
^{†}Different from placebo on Tukey post-hoc

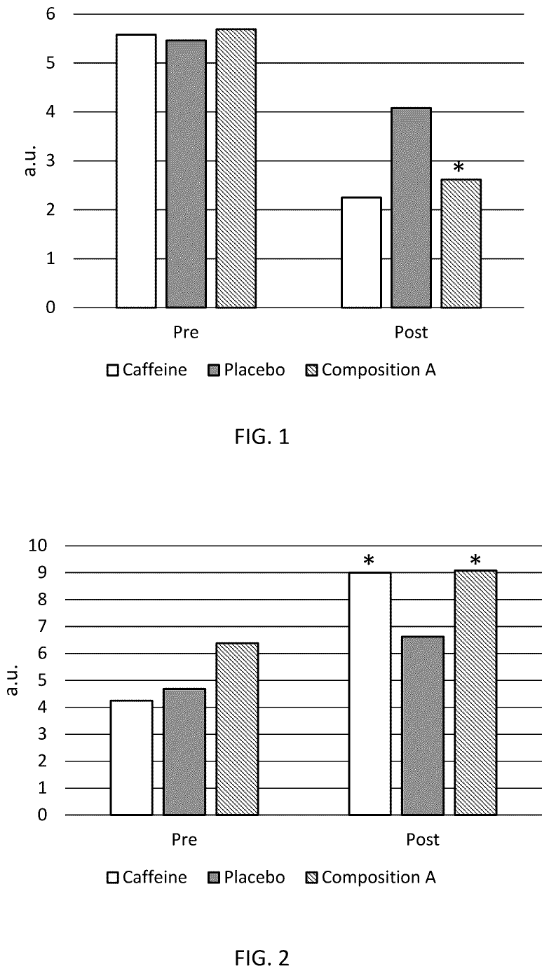

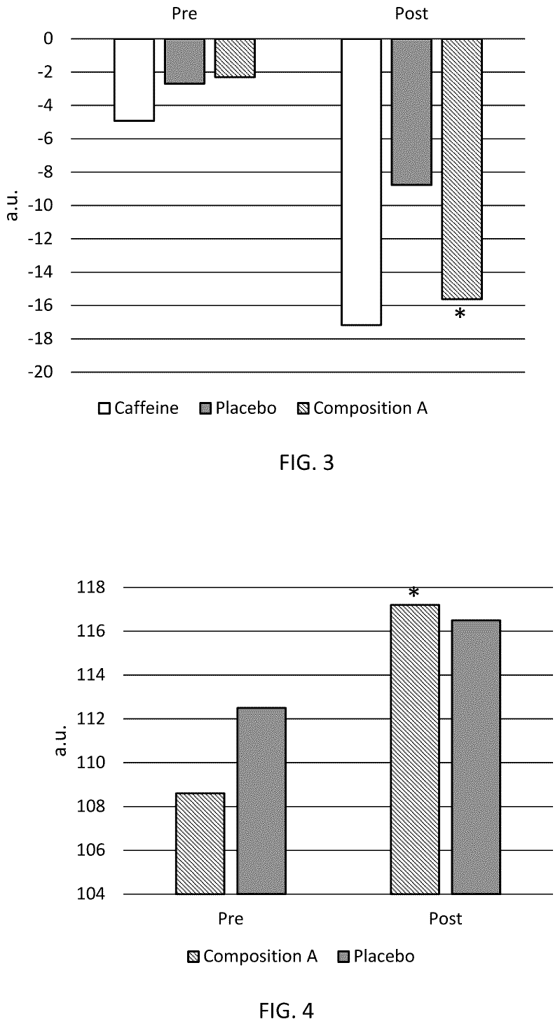

In caffeine-habituated users, the provision of caffeine alone did not relieve their feelings of fatigue, and only Caffeine and Composition A improved their vigor (energy). See Tables 16A and 16B. See also FIGS. 1-3.

TABLE 16A

| Variable | Caffeine | | Composition A | |
|---|---|---|---|---|
| (n = 12-13) | Pre | Post | Pre | Post |
| Tension | 3.00 ± 2.73 | 1.83 ± 3.54 | 4.08 ± 3.82 | 3.15 ± 3.51 |
| Anger | 1.42 ± 2.23 | 1.33 ± 3.45 | 2.38 ± 3.55 | 0.54 ± 1.45 |
| Fatigue | 5.58 ± 4.70 | 2.25 ± 3.02 | 7.31 ± 3.84 | 2.62 ± 3.64* |
| Depression | 1.50 ± 3.45 | 1.17 ± 4.04 | 2.00 ± 4.06 | 1.00 ± 2.38 |
| Esteem-related Affect | 14.42 ± 3.23 | 15.92 ± 3.15 | 15.31 ± 2.29 | 15.69 ± 2.75 |
| Vigor | 4.25 ± 2.93 | 9.00 ± 4.07* | 6.38 ± 3.12 | 9.08 ± 2.69* |
| Confusion | 2.25 ± 2.38 | 1.17 ± 2.92 | 3.62 ± 2.36 | 1.85 ± 1.57* |
| TMD | −4.92 ± 13.92 | −17.17 ± 17.69 | −2.31 ± 17.23 | −15.62 ± 13.25* |

TABLE 16B

| Variable | Composition B | | Placebo | |
|---|---|---|---|---|
| (n = 12-13) | Pre | Post | Pre | Post |
| Tension | 4.92 ± 3.90 | 2.85 ± 3.34 | 3.38 ± 3.04 | 2.69 ± 3.66 |
| Anger | 2.38 ± 4.57 | 1.15 ± 3.31 | 1.85 ± 3.02 | 1.54 ± 3.69 |
| Fatigue | 5.69 ± 4.15 | 3.38 ± 4.03 | 5.46 ± 3.73 | 4.08 ± 4.07 |
| Depression | 2.77 ± 5.05 | 1.43 ± 3.84 | 1.77 ± 3.49 | 1.92 ± 4.23 |
| Esteem-related Affect | 13.85 ± 2.58 | 14.54 ± 3.45 | 13.77 ± 3.11 | 14.23 ± 3.66 |
| Vigor | 4.62 ± 3.66 | 6.38 ± 4.52 | 4.69 ± 3.09 | 6.62 ± 3.97 |
| Confusion | 3.31 ± 3.71 | 2.00 ± 2.86 | 3.31 ± 3.01 | 1.85 ± 3.16 |
| TMD | 0.62 ± 17.97 | −10.08 ± 17.88 | −2.69 ± 15.86 | −8.77 ± 20.6 |

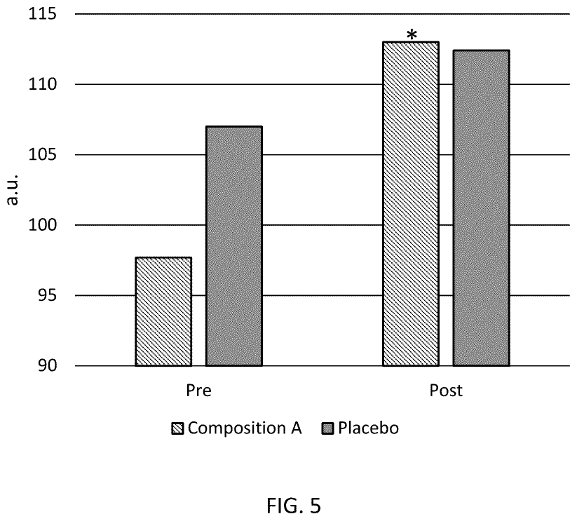

Additionally, among low-caffeine users, Composition A showed a significant improvement in both processing speed and executive function, while placebo showed no improvement. See FIGS. 4 and 5.

### Summary

Composition A improves/enhances/boosts processing speed, executive function, energy, focus, alertness, attention, memory, and mood (focus, alertness, attention, memory, and mood were improved among moderate caffeine users; processing speed and executive function were not tested in moderate caffeine users). Composition A also reduces/lessens/ameliorates fatigue and confusion (confusion was reduced among moderate-dose caffeine users). Composition A is fast acting and begins working within 60 minutes.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A brain health composition comprising a coffeeberry extract and one or more of caffeine, theacrine, methylliberine, and theanine.

2. The brain health composition of claim 1, wherein the brain health composition comprises the coffeeberry extract, caffeine, and two or more of theacrine, methylliberine, and theanine.

3. A brain health composition comprising:
caffeine;
a coffeeberry extract;
theacrine; and
methylliberine.

4. The brain health composition of claim 1, wherein the caffeine is present in an amount of about 60 mg to about 200 mg per serving of the brain health composition.

5. The brain health composition of claim 1, wherein the caffeine comprises instant release caffeine and sustained release caffeine.

6. The brain health composition of claim 5, wherein the instant release caffeine is present in an amount of about 60 mg to about 200 mg per serving of the brain health composition.

7. The brain composition of claim 5, wherein the instant release caffeine is anhydrous caffeine.

8. The brain health composition of claim 5, wherein the sustained release caffeine is present in an amount of about 50 mg to about 200 mg per serving of the brain health composition.

9. The brain health composition of claim 5, wherein the sustained release caffeine is released over a period of about 2 to about 12 hours when consumed by a mammal.

10. The brain health composition of claim 5, wherein the sustained release caffeine exhibits a release lag time of about 2 to about 4 hours followed by complete release by about 6 to about 8 hours when consumed by a mammal.

11. The brain health composition of claim 1, wherein the coffeeberry extract is present in an amount of about 50 mg to about 250 mg per serving of the brain health composition.

12. The brain health composition of claim 1, wherein the coffeeberry extract is a C. arabica fruit extract.

13. The brain health composition of claim 1, wherein the theacrine is present in an amount of about 40 mg to about 100 mg per serving of the brain health composition.

14. The brain health composition of claim 1, wherein the methylliberine is present in an amount of about 40 mg to about 100 mg per serving of the brain health composition.

15. The brain health composition of claim 1, wherein the brain health composition is formulated as a tablet, a capsule, or a soft gel.

16. The brain health composition of claim 15, wherein one tablet, capsule, or soft gel comprises a full serving of the brain health composition.

17. A method of improving one or more of alertness, reaction time, attention, mental accuracy, working memory, and video game performance in a mammal, comprising administering to the mammal the brain health composition of claim 1.

18. The method of claim 17, wherein one or more of alertness, reaction time, attention, mental accuracy, working memory, and video game performance are improved after administration of the brain health composition as compared to before administration of the composition.

19. A method of reducing one or more of fatigue, anxiety, occasional feelings of stress, and test errors in a mammal, comprising administering to the mammal the brain health composition of claim 1.

20. A method of supporting one or more of healthy energy levels, mood, and brain function in a mammal, comprising administering to the mammal the brain health composition of claim 1.
